(19) United States
(12) Patent Application Publication
(10) Pub. No.: US 2002/0009429 A1
(43) Pub. Date: Jan. 24, 2002

(54) PHARMACEUTICAL COMPOSITION COMPRISING A SELECTED ANTIGEN AND CANDIDA SPECIES ANTIGEN AND METHODS

(75) Inventor: EILEEN F. BOSTWICK, DAYTON, MN (US)

Correspondence Address: MERCHANT & GOULD PC, P.O. BOX 2903, MINNEAPOLIS, MN, 55402-0903, US

(73) Assignee: GalaGen, Inc., Arden Hills, MN

(21) Appl. No.: 09/240,249

(22) Filed: Jan. 29, 1999

(51) Int. Cl. A01N 63/00; A01N 65/00; A61K 39/00; A61K 39/38

(52) U.S. Cl. 424/93.1; 424/184.1

(57) ABSTRACT

The present invention provides compositions and methods for making and using antibodies. A pharmaceutical composition of the invention comprises a selected antigen and a Candida spp. antigen. Selected antigens include, for example, antigens derived from bacteria, yeasts, rickettsias, protozoas, viruses, parasites, and components or fragments thereof. Preferred compositions disclosed include antigens selected from Cryptosporidium spp. and Clostridium spp. The invention further provides for immunizing an animal with a composition of the invention to prepare a composition of immunoglobulins reactive with the selected antigen. The composition of immunoglobulins can be included in nutraceuticals that are ingested or administered enterally.

### BACKGROUND

Cryptosporidium spp. was once thought to be a commensal organism. However, in 1955 the organism was associated with turkey enteritis. Florence G. Crawford, “Human Cryptosporidiosis,” CRC Critical Reviews and Microbiol., 16 (2): 113-159, 113 (1988). The organism was later found to be a bovine pathogen in 1971 and a human pathogen in 1976. Id. Cryptosporidium spp. is now recognized as an important enteric protozoan pathogen, most commonly identified in cases of acute, self-limiting diarrheal diseases in poultry and mammals. Edward N. Janoff et al., “Cryptosporidium Species, a Protean Protozoan,” J. Clin. Microbiol., 25 (6):967-975, 970 (June 1987). The species which causes disease in humans is believed to be Cryptosporidium parvum. Id. at 113.

In cattle, Cryptosporidium is most commonly seen in calves less than three weeks old. “Cryptosporidiosis, in Current Veterinary Therapy: Food Animal Practice 779 (Jimmy L. Howard ed. 1990). The disease is accompanied by anorexia, dehydration, weight loss, debility and occasionally death. Id.

Although the precise prevalence of Cryptosporidium in humans is unknown, it is recognized worldwide as a common cause of enteritis. Rosemary Soave et al., “Cryptosporidium and Other Protozoa Including Isospora, Sarcocystis, Balantidium coli and Blastocystis,” in Principles and Practice of Infectious Diseases 235 (Gerald L. Mandel et al., eds., 1990). The organism is commonly found in immunocompetent patients showing clinical symptoms of diarrhea. Janoff at 967. Symptoms in humans include diarrhea, abdominal pain, cramping, vomiting, anorexia, malaise and weight loss and may include death in young children and aged adults. Id. at 971. The pathogenesis of human Cryptosporidium is not completely known. Crawford at 145; Janoff at 970.

The Cryptosporidium organism is also found in immunocompromised individuals. Today, many cases of Cryptosporidium in immunocompromised individuals are in persons suffering from acquired immunodeficiency syndrome (AIDS). In one study, the most common pathogen associated with diarrhea in AIDS patients was Cryptosporidium. Barbara E. Laughon et al., “Prevalence of Enteric Pathogens in Homosexual Men With and Without Acquired Immunodeficiency Syndrome,” Gastroenterology, 94(4):984-992, 984 (April 1988). Moreover, unlike the symptoms seen in immunocompetent patients, the syndrome in immunocompromised individuals may be of greater severity and may persist for many months causing anorexia, abdominal pain, weight loss, vomiting, diarrhea, malaise, low-grade fever, and even death due to dehydration and cachexia. Janoff at 971. In addition, occasional coughing and progressive pulmonary disease are seen. Id. at 971.

Therefore, as seen in immunocompromised individuals, Cryptosporidium is not necessarily self-limiting. Id. In fact, CDC sources have reported that cumulative case fatality rates through April 1986 were significantly higher in AIDS patients affected by Cryptosporidium. Crawford at 132. Moreover, it is believed that AIDS patients who recover from clinical cryptosporidiosis still harbor low levels of Cryptosporidium oocysts. Id.

In humans, treatment of Cryptosporidium using single and multiple-drug regimens has, at best, met with limited success. Janoff at 972; Crawford at 147; K. W. Angus, “Cryptosporidiosis and AIDS,” Bailliere's Clinical Gastroenterology 4(2):425-441, 435 (June 1990). And, while immunoprophylaxis has been suggested, a Cryptosporidium vaccine capable of producing immune stimulation has not been described. Angus at 436-37.

Clostridium difficile was first described in 1935. Although the organism released potent toxins in broth culture, it was also found in stool specimens of healthy infants. Thus, Clostridium difficile was labeled a commensal organism and was not studied further until Clostridium difficile was linked to antibiotic-associated pseudomembranous colitis (PMC), a gastrointestinal illness, in the 1970s. Kelly et al., “Clostridium Difficile Colitis,” N. Eng. J. of Med., 330: 257-262, 257 (1994). Gastrointestinal health greatly depends on the normal bacterial flora in the colon. The normal flora is a barrier against colonization by pathogens, and the disruption of this flora in a host results in the host becoming susceptible to colonization or overgrowth of a pathogen. Lyerly et al., “Clostridium Difficile: Its Disease and Toxins,” Clin. Micro. Rev., 1: 1-18, 3 (1988). Antibiotics disrupt the normal intestinal flora of the patient and lead to the patient being susceptible to colonization with Clostridium difficile. Such disruptive antibiotics include clindamycin, ampicillin, penicillins, and cephalosporins. Yet almost any antibiotic can result in intestinal colonization with Clostridium difficile, leading to the release of Clostridium difficile toxins that cause mucosal damage and inflammation. Id.; Kelly et al. at 257.

Clostridium difficile is a troublesome organism because it forms heat-resistant spores that allow the organism to remain a viable infectious agent for months and even years. Kelly et al. at 257. As a result, Clostridium difficile can be a widespread contaminate. In particular, environmental contamination of these spores is commonly found in hospitals and long-term care facilities. In fact, several reports of hospitals and nursing homes have identified Clostridium difficile infection as having been epidemic or endemic. Bartlett, “Antibiotic-Associated Diarrhea,” Clin. Infectious Diseases, 15: 573-81, 575-76 (1992). Clostridium difficile infection arises from oral ingestion of the spores, which survive the acid environment of the stomach and convert to vegetative forms in the colon. Kelly et al. at 257. Once established in the colon, pathogenic strains of Clostridium difficile produce toxins that cause diarrhea, colitis, mucosal damage, and inflammation. Id. Two large protein exotoxins are produced by Clostridium difficile: toxin A (a 308 kDa enterotoxin) and toxin B (a 250-270 kDa cytotoxin). Id. The susceptibility of the human intestine to the effects of the two toxins has not been extensively investigated, but preliminary studies indicate that the colon may be vulnerable to both. Id.

Clostridium difficile infection primarily takes three forms: diarrhea, severe colitis without pseudomembrane formation, and pseudomembranous colitis. Kelly et al. at 259. If infection causes mild to moderate diarrhea and no more than lower abdominal cramping, the problem usually subsides by terminating antibiotic use, in which case no specific treatment for Clostridium difficile is required. Id. Severe colitis without pseudomembrane formation may occur with profuse, debilitating diarrhea, abdominal pain, and distention. Common systemic manifestations include fever, nausea, anorexia, malaise, and dehydration. Occult colonic bleeding may also occur. Id. Pseudomembranous colitis leaves patients acutely ill, with lethargy, fever, tachycardia, and abdominal pain. Colonic muscular tone also may be lost, resulting in toxic dilation or megacolon. Id.

Clostridium difficile also may cause other diseases, including abscesses, wound infections, osteomyelitis, pleuritis, peritonitis, septicemia, and urogenital tract infections. Lyerly et al. at 4.

Treatment of Clostridium difficile includes discontinuing use of the implicated antibiotic and administering a specific means of treatment. Bartlett, Clin. Infectious Diseases, 15: 573-81, 578 (1992). The symptoms of Clostridium difficile-associated diarrhea or colitis may persist for weeks or months after the use of the implicated antibiotic is terminated. Id. at 575. Problems also arise in using specific means of treating Clostridium difficile infection because the drugs with effective activity against the organism, including ampicillin and vancomycin, may actually induce Clostridium difficile-associated illness. Id. at 573.

Clostridium difficile continues to infect millions of patients each year and continues to pose a diagnostic and therapeutic challenge. Kelly et al. at 257. The spread of Clostridium difficile in hospitals is a major concern and demands that preventive measures be taken. U.S. Pat. No. 5,773,000 to Bostwick et al. teaches the effective treatment of Clostridium difficile-associated diseases by administering an antibody having specific activity against Clostridium difficile.

The use of adjuvants to enhance in vitro immune stimulation against various organisms is well known. Adjuvants known in the art include, for example, alum, aluminum hydroxide, aluminum phosphate, and water-in-oil emulsions. In addition, known adjuvants may include components of microorganisms as immuno-stimulants; for example, Freund's-complete-adjuvant is a water-in-oil adjuvant which also contains dead Mycobacteria. Other species of bacteria are also known to enhance the immune response of a human or animal, for example, Nocardia, Bordetella, and Corynebacterium parvum.

The use of Candida spp. antigens to stimulate specific immunity to the Candida organism is known in the art. However, the use of Candida antigens as an adjuvant material to enhance the in vitro immune response to bacterial antigens has not been described.

### SUMMARY OF THE INVENTION

The present invention provides a pharmaceutical composition for enhancing the immune response of an animal against a selected antigen. Specifically, a composition can be composed of a selected antigen and a Candida spp. antigen. According to the invention, a selected antigen can be derived from bacteria, yeasts, rickettsias, protozoas, viruses, parasites, etc., or components or fragments thereof. In one embodiment, the selected antigen is Cryptosporidium parvum in combination with a Candida albicans antigen. In another embodiment, the selected antigen can be a Clostridium difficile antigen in combination with an antigen derived from Candida albicans. The compositions of the invention may additionally contain an adjuvant known in the art. The pharmaceutical compositions may be used to vaccinate or immunize an animal against a selected antigen.

The invention further provides a method for enhancing the immune response of an animal against a selected antigen by administering a pharmaceutical composition containing a selected antigen and a Candida spp. antigen. The method provides for administration of one or more compositions of the invention through oral, subcutaneous, intramuscular, intradermal, intramammary, intravenous, or other administration methods known in the art.

Another aspect of the invention provides an immunoglobulin composition prepared from an animal immunized or vaccinated with a pharmaceutical composition of the present invention.

In yet another aspect, the invention provides a nutraceutical comprising an immunoglobulin composition collected from an animal wherein the immunoglobulin composition includes immunoglobulins that are reactive with an antigen against which the animal was immunized. The invention also provides methods for preparing a nutraceutical.

### DETAILED DESCRIPTION

The present invention provides pharmaceutical compositions and methods for preparing compositions that enhance an animal's immune response against a selected antigen. This includes antigens which typically may not stimulate a strong immune response due to, for example, poor antigen recognition by an animal's immune system. The invention also provides a method for using a composition of the invention to enhance the immune response of an animal against a selected antigen by administering the composition of the invention to an animal through methods commonly used in the art.

As used herein, the term “animal” includes mammals such as humans, mice, cattle, goats, sheep, guinea pigs, rabbits, etc., and nonmammals such as avians, including, for example, chicken, turkeys, ducks, geese, etc.

It will be noted that at several places throughout the present specification, guidance is provided through lists of examples. In each instance, the recited lists serve only as representative groups. It is not meant, however, that the lists are exclusive. Also, it must be noted that, as used in this specification and the appended claims, the singular forms “a,” “an,” and “the” include plural referents unless the content clearly dictates otherwise. Thus, for example, reference to a composition containing “a microorganism” includes a mixture of two or more microorganisms and reference to “an immunoglobulin” includes two or more immunoglobulins.

As used herein, an “immunizing composition” means a composition that stimulates an animal's immune response (either or both the humoral or cellular immune response) against an antigen. According to the invention, the herein disclosed compositions are prepared by combining a selected antigen and a Candida spp. antigen. Although the inventors do not wish to be limited to a single mechanism, it is believed that when administered to an animal, a Candida spp. antigen can enhance the immune response to a selected antigen which is combined with the Candida spp. antigen. This includes an enhanced response against poorly recognized antigens. The immune enhancing affect of a Candida spp. antigen can be in either or both of the primary and secondary (anamnestic) immune response.

As used herein, the term “antigen” means a substance or entity that is structurally or functionally capable of inducing an immune response in an animal. This includes antigens which typically produce only a very poor immune response. According to the invention, an “antigen” includes, but is not limited to, inactivated whole microorganisms, attenuated whole microorganisms, whole viral particles, antigenic microorganism/viral components or fragments, chemically or physically modified antigens, recombinant antigens, and other antigens or combinations thereof known and used in the art.

In one embodiment, the selected antigen which is combined with the Candida spp. antigen is derived from Cryptosporidium spp. One aspect to a composition of the present invention is the unexpected discovery that a Candida spp. antigen combined with a selected antigen enhances an animal's immune response against the selected antigen in the absence of other adjuvants. However, the invention also provides for combining the selected antigen and a Candida spp. antigen with one or more adjuvants known in the art which may further enhance the immune response.

As used herein, a Candida spp. antigen may be a whole Candida spp. organism in any of its forms (e.g., hyphal form, budding form, etc.), inactivated whole organism, fragments or components isolated from the whole organism or specific Candida spp. antigens produced through genetic engineering methods known in the art. Preferably, the Candida spp. antigen of the invention is prepared by inactivation of a live Candida spp. organism. Methods of inactivation useful according to the invention, include, for example, formaldehyde inactivation, heat treatment, hypochlorite inactivation, irradiation, and other methods known in the art. Also, if the immunizing composition is combined with one or more adjuvants known in the art, the inventors recognize that many of the known adjuvants may inactivate the Candida spp. organism without the Candida spp. first being inactivated by the above recited methods.

A selected antigen of the invention against which immunity is desired may be prepared by methods commonly used in the art. As used herein, a selected antigen may be a whole organism in any of its life cycle stages, inactivated whole organism, fragments or components isolated from the whole organism, specific antigens genetically engineered through methods known in the art or other antigens as defined earlier in this disclosure. In addition, the selected antigen can be derived from either or both a mature whole organism or sporozoites (oocysts). Preferred selected antigens of the invention include, for example, antigens derived from bacteria, yeasts, protozoas, viruses, rickettsias, parasites such as a helminths, and fragments or components thereof. Examples of fragments or components isolated from a whole organism, include, but are not limited to, toxins from the organism and cell surface antigens.

One selected antigen of the invention is derived from the protozoan, Cryptosporidium parvum. According to this embodiment, a composition can be prepared by combining the Candida spp. antigen with the selected antigen. When using an inactivated whole cell Candida spp. antigen, the number of cells in a single mammalian dose of vaccine is about 2×10³ to 2×10¹¹, preferably 2×10⁶ to 2×10⁹. The amount of a selected antigen to be administered to an animal in a single dose of the composition will vary with the selected antigen and can be readily quantitated by one of skill in the art. When the selected antigen is Cryptosporidium spp. a single animal dose of a composition may contain 2×10⁴ to 2×10¹² oocysts, preferably 2×10⁵ to 2×10⁹ oocysts.

Although it is not deemed necessary, a composition of the invention containing a selected antigen and Candida spp. antigen may be further combined with a pharmaceutically acceptable carrier. Pharmaceutically acceptable carriers useful according to the invention include physiological saline, ringers, lactated ringers, phosphate buffered saline, and other carriers known in the art.

In another embodiment, a composition of the invention may include a selected antigen, Candida spp. antigen, and one or more adjuvants selected from adjuvants known in the art. When an adjuvant is mixed with the Cryptosporidium and Candida spp. antigen, the adjuvant can be mixed with the combined antigens in a volume/volume (v/v) ratio of 3:1 to 1:5, preferably 1:1. Adjuvants known in the art which are suitable for the invention include, but are not limited to, incomplete Freund's adjuvant (IFA), Freund's complete adjuvant, saponins, Quil A, mineral oil, aluminum hydroxide, aluminum phosphate, muramyl dipeptide, block copolymers, and synthetic polynucleotides.

The present invention further provides a method for enhancing the immunity of an animal to a selected antigen by administering a composition of the invention to an animal through methods known in the art. Such methods of administration include enteral administration and parenteral administration including subcutaneous, intramuscular, intradermal, intramammary, and intravenous administration. Typical immunization methods include intramuscular and subcutaneous administration.

The enhanced immunity to selected antigens provided by administering a composition of the invention was studied using animal models. Mouse inoculation studies using a selected antigen of Cryptosporidium parvum and a Candida albicans antigen, regardless of the presence or absence of additional adjuvants, provided significantly higher Cryptosporidium parvum serum immunoglobulin titers than when Candida albicans antigen was omitted. Calf inoculation studies using a Cryptosporidium parvum antigen and a Candida albicans antigen, regardless of the presence or absence of the additional adjuvants, also produced a significant increase in Cryptosporidium parvum serum immunoglobulin titers over titers produced in calves inoculated with the Cryptosporidium parvum antigen alone. The enhanced immune response was detectable after a primary or a secondary immunization with a composition of the invention.

The present invention is also directed to pharmaceutical compositions comprising a bacterial antigen and a Candida spp. antigen, to methods for preparing the compositions, and methods of using the compositions. A bacterial antigen of the invention can be derived from a gram-positive bacterium, including Clostridia, Staphylococci, Streptococci, etc. and/or gram-negative bacterium, including Klebsiella, Escherichia coli, Salmonella, etc. As discussed above, one or more adjuvants known in the art also can be included in the composition. The immune enhancing effect of a Candida spp. antigen in the bacterial antigen containing compositions of the invention is independent of the absence or presence of additional adjuvants. The Candida spp. antigen may be derived from any Candida spp. including, for example, Candida glabrata, Candida krusei, and Candida albicans.

In one embodiment, the selected bacterial antigen that is combined with the Candida spp. antigen is derived from Clostridium difficile. In one embodiment, the Clostridium difficile antigen can be toxoided. A toxoided Clostridium difficile antigen comprises a toxoided culture of Clostridium difficile. A toxoided culture comprises an inactivated whole cell homogenate of Clostridium difficile that includes toxins A and B and cell surface antigens. In a presently preferred embodiment, the toxoided culture of Clostridium difficile comprises a whole cell homogenate of the Clostridium difficile organism deposited under ATCC accession no. 202193. Alternatively, a selected antigen may include fragments or components isolated from a Clostridium difficile organism, for example, toxin A, toxin B, or cell surface antigens. Fragments or components may also be isolated from the Clostridium difficile organism deposited under ATCC accession no. 202193, or any other Clostridium difficile organism.

Thus, in one embodiment, a pharmaceutical composition can be prepared by combining a Candida spp. antigen with a selected bacterial antigen. When using an inactivated whole cell Candida spp. antigen, the number of cells in a single mammalian dose of vaccine typically can be about 2×10³ to 2×10¹¹, preferably 2×10⁶ to 2×10⁹. In some preferred embodiments, the Candida spp. antigen is Candida albicans. The amount of a selected antigen to be administered to an animal in a single dose of the composition will vary with the selected antigen and can be readily quantitated by one of skill in the art. When the selected antigen is toxoided Clostridium difficile, a single dose of a pharmaceutical composition may contain 0.5 to 5.0 ml of toxoided, concentrated, culture filtrate, preferably 1.0 to 3.0 ml.

If an adjuvant is mixed with the Clostridium and Candida spp. antigen, the adjuvant is mixed with the combined antigens in an antigen volume/adjuvant volume (v/v) ratio of about 3:1 to 1:5, typically about 1:1. Adjuvants known in the art which are suitable for the invention are listed above.

The present invention also provides an immunoglobulin composition comprising immunoglobulins collected from an animal administered a pharmaceutical composition of the invention. Such an immunoglobulin composition can be prepared from antibodies or immunoglobulins collected from an animal after administration of a pharmaceutical composition of the invention. Typically, antibodies suitable for the immunoglobulin composition are collected one to four weeks after administration of the pharmaceutical composition. In the case of an animal that has never been immunized against the selected antigen, a booster administration is recommended after initial immunization using known protocols. The immunoglobulins can be collected from the blood, serum, plasma, or milk of the animal. A preferred source of the immunoglobulin composition is bovine colostrum.

Another aspect of the invention provides a nutraceutical or functional food comprising an immunoglobulin composition from an animal wherein the immunoglobulin composition includes enhanced levels of immunoglobulins reactive with an antigen against which the animal has been selectively immunized. As used herein, a “nutraceutical” or “functional food” means modified food or food ingredient that can provide a health benefit beyond the benefit that nutrients the food or food ingredient typically contains. Nutraceuticals may include dietary supplements, medical foods, consumer foods, and infant formulas. Nutraceuticals may be ingested or administered orally or enterally. Forms for ingestion or administration include a tablet, capsule, powder, liquid, sports drink, candy bar, etc.

Nutraceuticals can help to maintain the structure and function of the human body, for example, supporting the body's natural microflora. When consumed, a nutraceutical including an immunoglobulin composition of the present invention may also maintain or support health in the presence of, for example, a diarrheal disease caused by organisms such as Clostridium difficile or Cryptosporidium parvum.

Known methods for preparing tablets and capsules including immunoglobulins according to the invention can be used. For example, tablets can be prepared by combining an immunoglobulin composition with conventional excipients, binders and disintegrates, including, for example, polyvinyl pyrrolidone, sodium citrate, calcium carbonate and dicalcium phosphate, starch, alginic acid, complex silicates, milk sugar, gelatin, acadia, etc. Additionally, lubricating agents such as magnesium stearate, sodium laurel sulfate and talc are often useful for tableting purposes. Immunoglobulin compositions may also be formulated into oral gelatin capsules, including excipients such as, lactose or milk sugar, as well as high molecular weight polyethylene glycols.

Nutraceuticals can include components to fortify the nutritional or health benefit of the composition or enhance consumer acceptance through natural or artificial flavoring or coloring. Examples of compositions for promoting and maintaining gastrointestinal health are disclosed in U.S. Pat. Nos. 5,531,988, 5,531,989, and 5,744,134, the entire disclosures of each being incorporated herein by reference.

For example, a nutraceutical can include yogurt cultures or kefir cultures to replenish or enhance normal gastrointestinal flora. Bacterial organisms suitable for a yogurt culture include, for example, Streptococcus thermophilus and bacteria of the Lactobacillus and Bifidobacterium genera, such as, L. acidophilus, L. bulgaricus, L. casei L. fermentum, L. salivaroes, L. brevis, L. leichmanii, L. plantarum, L. cellobiosus, B. infantis, B. longum, B. thermophilum and B. bifidum.

Kefir cultures for production of a fermented milk product may contain a mixture of symbiotic yeast, lactobacilli, leuconostocs, and lactic streptococci.

Vitamins and minerals can also be added to enhance the nutritional benefits provided by a nutraceutical of the invention. Vitamins include fat soluble and water soluble vitamins, and minerals include macro and micro minerals. Soluble fiber may also be added.

In addition, natural fruits, fruit juices or fruit seeds can be included for flavor, texture and added nutritional benefit. Suitable fruits and fruit seeds includes, for example, banana, pineapple, apple, orange, peach, strawberry, cherry, raspberry, blueberry, kiwi, nuts, and rice.

In addition, artificial flavoring and colors can be added to enhance and accommodate consumer acceptance and preferences. FDA approved artificial flavorings and colorings for use in food products are known and suitable for a nutraceutical of the invention.

The immunoglobulin composition included in a nutraceutical may be obtained from any method of antibody preparation suitable for human ingestion or enteral administration. In one embodiment, gestating cows are immunized with a pharmaceutical composition comprising Clostridium difficile and a Candida spp. antigen. After parturition, the colostral milk can be collected according to the method disclosed in U.S. Pat. No. 5,773,000 to Bostwick et al. In another embodiment, the immunoglobulin composition can be collected from the eggs of an avian host that was administered a pharmaceutical composition of the invention. Methods for immunizing avian hosts and collecting antibodies are known and include, for example, U.S. Pat. No. 5,601,823 to Williams et al.

Additional nutraceutical compositions suitable for use with an immunoglobulin composition of the present invention are disclosed in co-pending application Ser. No. 60/105,649, filed Oct. 26, 1998, the entire disclosure of which is incorporated herein by reference.

The following examples describe preparation and administration of compositions of the invention.

### EXAMPLES

### Example 1

Preparation of Cryptosporidium parvum and Candida albicans Vaccine Compositions

Cryptosporidium parvum vaccine compositions were prepared with multiple concentrations of Cryptosporidium parvum antigen and multiple concentrations of Candida albicans antigen. Vaccine compositions containing Cryptosporidium parvum antigens, Candida albicans antigen and various additional adjuvants were also prepared and tested.

Vaccine compositions were prepared to provide 8×10⁶ Cryptosporidium oocysts and 2×10⁷ Candida albicans cells per dose of mouse vaccine and 3×10⁸ Cryptosporidium oocysts and 2×10⁷ Candida albicans cells per dose of calf vaccine.

The vaccine compositions were prepared by combining Cryptosporidium parvum oocysts with Candida albicans cells in a ratio of about 1:1 to 1:10, preferably 1:3 to 1:6. The Cryptosporidium parvum oocysts and the Candida albicans cells were counted using a hemocytometer (Hausser Scientific, Horsham, Pa.). The Cryptosporidium parvum antigen was prepared using three cycles of freezing and thawing of Cryptosporidium parvum oocysts. The Candida albicans antigen was prepared by adding 1% formaldehyde of Candida albicans cells to a final concentration of 0.37%.

The additional adjuvant used in some vaccine compositions was incomplete Freund's adjuvant (IFA) and mineral oil. Phosphate buffered saline (PBS) was used as a control. The Cryptosporidium parvum and Candida albicans preparations as described above were combined with an adjuvant in a 1:1 v/v ratio. Vaccine compositions containing mineral oil were prepared by simply mixing the mineral oil with the Cryptosporidium and Candida albicans antigen combination. Vaccine compositions containing IFA were emulsions prepared by mixing IFA with the Cryptosporidium and Candida albicans antigen combination followed by sonication with a microprobe at 25 watts for 30 seconds at 100% power. PBS, mineral oil and IFA are commonly available to those skilled in the art.

Some of the various vaccine combinations prepared are shown in Table 1. The table also shows antigen quantities used per immunizing dose.

TABLE 1

| | |
|---|---|
| Antigen Quantities per Immunization | Adjuvants |

MOUSE VACCINE COMPOSITIONS

| | | | |
|---|---|---|---|
| 1 | 8 × 10⁶ Crypto | PBS | Incomplete Freund's Adjuvant |
| 2 | 8 × 10⁶ Crypto | 2 × 10⁷ Candida | Incomplete Freund's Adjuvant |
| 3 | 8 × 10⁶ Crypto | PBS | Mineral Oil |
| 4 | 8 × 10⁶ Crypto | 2 × 10⁷ Candida | PBS |
| 5 | 8 × 10⁶ Crypto | 2 × 10⁷ Candida | Mineral Oil |

CALF VACCINE COMPOSITION

| | | | |
|---|---|---|---|
| 1 | 3 × 10⁸ Crypto | 2 × 10⁹ Candida | Incomplete Freund's Adjuvant |
| 2 | 3 × 10⁸ Crypto | 2 × 10⁹ Candida | PBS |
| 3 | 3 × 10⁸ Crypto | PBS | Incomplete Freund's Adjuvant |
| 4 | PBS | 2 × 10⁹ Candida | Incomplete Freund's Adjuvant |

### Example 2

Preparation of Mouse Vaccine

To prepare a mouse vaccine composition, 5 ml of 8.0×10⁷/ml Cryptosporidium parvum oocytes were combined with 5.0 ml of 2×10⁸/ml Candida albicans cells. If an additional adjuvant was used, 1.0 ml of the combined Cryptosporidium parvum and Candida albicans antigens were mixed with 1.0 ml of adjuvant.

The 5.0 ml of 8.0×10⁷/ml Cryptosporidium parvum oocysts were prepared by mixing 0.075 ml Cryptosporidium parvum oocysts with 4.925 ml PBS. The 5.0 ml 2×10⁸/ml Candida albicans cells were prepared by mixing 1 ml of 1×10⁹/ml Candida albicans cells with 4 ml PBS. The final relative concentration of the combined Cryptosporidium and Candida albicans antigens was 4×10⁷/ml and 1×10⁸/ml, respectively.

If adjuvant was used, 1.0 ml of the mixed Cryptosporidium and Candida albicans antigen was mixed with 1.0 ml of adjuvant. When mineral oil was used as an adjuvant, the adjuvant was combined with the antigens by simple mixing. When IFA was used, an emulsion was formed using a microprobe as described in Example 1. Therefore, the mouse vaccine composition contained 2×10⁷/ml Cryptosporidium parvum oocysts and 5×10⁷/ml Candida albicans cells.

### Example 3

Preparation of Calf Vaccine

To prepare a calf vaccine composition, 50 ml of 6.4×10⁸/ml Cryptosporidium parvum oocysts were combined with 50 ml of 4×10⁹/ml Candida albicans cells. If an additional adjuvant was used, 1.0 ml of the combined Cryptosporidium parvum and Candida albicans cells was mixed with 1.0 ml of adjuvant.

The 50 ml of 6.4×10⁸/ml Cryptosporidium parvum oocysts/sporozoites were prepared by mixing 16 ml of 2×10⁹/ml Cryptosporidium parvum oocysts with 34 ml PBS. The 5.0 ml of 4×10⁹/ml Candida albicans cells was prepared by mixing 40 ml of 5×10⁹/ml Candida albicans cells with 10 ml PBS. The final relative concentrations of the combined Cryptosporidium parvum and Candida albicans antigens were 3.2×10⁸/ml and 2×10⁹/ml, respectively.

If adjuvant was used, 1.0 ml of the mixed Cryptosporidium and Candida albicans antigen was mixed with 1.0 ml of adjuvant. When mineral oil was used as an adjuvant, the antigens and adjuvant were simply mixed together. When IFA was used, an emulsion was formed using a microprobe as described in Example 1. Therefore, the calf vaccine composition contained 1.6×10⁸/ml Cryptosporidium parvum oocysts and 1×10⁹/ml Candida albicans cells.

### Example 4

Mouse Immunization Studies

Mice were immunized with vaccine compositions prepared as described in Examples 1 and 2. Balb\c mice 6-8 weeks of age were used for the immunization study. Mice were divided into immunization groups with 4 mice in each group. Vaccine was administered subcutaneously by holding each mouse behind the head in a manner to leave the skin behind and below the shoulder blades as loose as possible. A total of 400 μl per mouse was administered using a 22 gauge needle. Hence, each 400 μl dose contained 8×10⁶/ml Cryptosporidium parvum oocysts and 2×10⁷/ml Candida albicans cells. A total of 3 immunization doses were given at approximately 2-week intervals.

Tail bleeds were performed at approximately 6½ weeks to obtain serum samples for testing antibody response. The tip (about 1 mm) of the tail was cut off using a sharp razor blade. Approximately 50 μl of blood was collected into 1.5 ml microcentrifuge tubes and allowed to coagulate overnight at 4° C. Samples were then spun in a microcentrifuge for 3 minutes at approximately 14,000× g to separate the serum from the clot. ELISA assays were used to determine serum titers of the mice to both Cryptosporidium parvum and Candida albicans antigens.

Pooled serum samples were also tested. At about 7 weeks the mice were bled out to collect a larger volume of serum. The mice were first anesthetized with a mixture of tribromoethanol and tert-amyl alcohol and ocular bleeds were performed. The blood was collected into microcentrifuge tubes. Serum was separated in the same manner as the tail bleeds described above. Six serum pools representing the six immunization groups were then made by combining 200 μl of serum from the 4 individual mice of each group. The pooled serum from the 6 immunization groups were titered in ELISA assays to both Cryptosporidium parvum and Candida albicans antigens. As shown below, titers from the serum pools were similar to the mathematical averages of the individual serum samples from each immunization group. Candida albicans titers over non-immune titers was also assayed on pooled samples and were determined to be unaffected by the presence of Cryptosporidium parvum antigens (i.e., adjuvant effect was one-way).

TABLE 2
MICE IMMUNIZATION

| | Cryptosporidium |
|---|---|
| | parvum Titers |
| | (Titers Over Non-Immune Serum) |
| Immunization Group | Mouse Number |

| | Crypto | Candida | Adjuvant | 1 | 2 | 3 | 4 | POOL |
|---|---|---|---|---|---|---|---|---|
| 1 | X | — | IFA | 800 | 400 | 400 | 400 | 800 |
| 2 | X | X | IFA | 12800 | 800 | 25600 | 1600 | 12800 |
| 3 | X | — | Mineral | 400 | 200 | 200 | 400 | 400 |
| | | | Oil | | | | | |
| 4 | X | X | — | 1600 | 800 | 800 | 800 | 800 |
| 5 | X | X | Mineral | 1600 | 400 | 800 | 3200 | 1600 |
| | | | Oil | | | | | |

### Example 5

Calf Immunization Studies

Calves were immunized with vaccine compositions prepared as described in Examples 1 and 3.

Four to six month old Holstein steer calves were used for this immunization study. All calves were healthy and, prior to the study, were treated with vitamin E-selenium, vitamin B complex and ivermectin. Calves were individually identified with 2 means of permanent identification and boostered with a killed vaccine preparation against IBR, PI₃, BRSV and BVD (Elite 4, Bio-Ceutic Laboratories, St. Joseph, Miss.). All calves were fed free choice hay supplemented with a balanced grain ration containing a coccidiostat (decoquinata).

The calves were randomly assigned to treatment groups of four (4) to five (5) calves per group. The calves were allowed to commingle during the study.

Each vaccine composition was administered intramuscularly. A total of 2 ml of the vaccine composition of Examples 1 and 3 was administered 4 times at 2-week intervals. Hence, each 2 ml dose contained 3.2×10⁸/ml Cryptosporidium parvum oocysts and 2×10⁹/ml Candida albicans cells.

The injection site of each inoculation was observed at the time of inoculation, at 24 hours post-inoculation and at weekly intervals for the duration of the study. No adverse systemic reactions were noted. Induction of a significant immune response was frequently associated with an unacceptable localized inflammatory response when an additional adjuvant was used.

Venous blood samples were taken at the time of each immunization and up to four weeks after the final injection to obtain serum samples for assessing antibody response. Approximately 20 ml of blood was collected into sterile tubes and allowed to coagulate overnight at 4° C. Samples were then spun in a centrifuge for 3 minutes at approximately 14,000× g to separate the serum from the clot. ELISA assays were used to determine serum titers of the calves to both Cryptosporidium parvum and Candida albicans antigens.

TABLE 3

| | Cryptosporidium Titers |
|---|---|
| | (Calf Titers Over |
| | Non-Immune Serum) |
| Immunization Group | Calf Number |

| | Crypto | Candida | Other | 1 | 2 | 3 | 4 | 5 | Average |
|---|---|---|---|---|---|---|---|---|---|
| 1 | X | X | IFA | 64 | 128 | 64 | 64 | 64 | 80 |
| 2 | X | X | PBS | 16 | 128 | 32 | 128 | — | 76 |
| 3 | X | — | PBS | 32 | 64 | 32 | 32 | — | 40 |

The potentiation of the immune response stimulated by a composition of the invention was evident even after primary immunization. Preimmunization titers were calculated relative to a non-immune control pooled serum standard.

TABLE 4

| | | | |
|---|---|---|---|
| | | Post Primary | |
| Calf Number | Preimmunization Titer | Immunization Titer | Increase |

VACCINE COMPOSITION USED: CRYPTO + CANDIDA + IFA

| | | | |
|---|---|---|---|
| 101 | 0.75 | 8 | 10.6X |
| 108 | 0.75 | 16 | 21.3X |
| 115 | 1.5 | 4 | 2.7X |
| 122 | 0.75 | 8 | 10.6X |
| 129 | 0.5 | 8 | 16X |

VACCINE COMPOSITION USED: CRYPTO + IFA

| | | | |
|---|---|---|---|
| 110 | 2 | 2 | — |
| 117 | 0.25 | 1 | 4X |
| 124 | 0.5 | 1 | 2X |
| 005 | 2 | 4 | 2X |

### Example 6

Preparation of Clostridium difficile and Candida albicans Composition

Toxoided Clostridium difficile was prepared from a culture filtrate (from Clostridium difficile deposited under ATCC accession no. 202193) according to methods described in U.S. Pat. No. 5,773,000. The culture filtrate contained high levels of toxins A and B and soluble cell surface antigens. For the culture filtrate, the strain was grown in brain heart infusion dialysis flasks at 37° C. for 72 hours as described in Sullivan et al., Infect. Immun., 35: 1032-40 (1982). The culture filtrate was converted to a toxoid by adding 37% formalin to a final volume of 1% v/v (0.37% formaldehyde) and incubating the mixture at 37° C. for 1 to 2 hours. The toxoided Clostridium difficile was then concentrated ten-fold by membrane ultrafiltration using a stirred cell apparatus equipped with a 10,000 kDa molecular weight cutoff depth filter under 20-25 psi nitrogen. The resulting toxoided antigen was evaluated by the USP (United States Pharmacopeia) method for sterility.

Two different pharmaceutical compositions using the toxoided antigen will be described. A first composition is prepared including a Clostridium difficile antigen without the Candida albicans antigen by vortexing a ten-fold concentrated, sterile toxoided Clostridium difficile antigen with an equal volume of sterile phosphate buffered saline (PBS) to a final volume of 1.5 ml. A second composition is prepared including a Clostridium difficile antigen and a Candida albicans antigen by vortexing a ten-fold concentrated, sterile toxoided Clostridium difficile antigen with an equal volume of sterile PBS to a volume of 1.5 ml. Then, 1.5 ml of whole cell Candida albicans antigen preparation is added to the Clostridium difficile preparation.

### Example 7

Preparation of Clostridium difficile and Candida albicans Composition with Incomplete Freund's Adjuvant

To prepare a composition of Clostridium difficile and Candida albicans with incomplete Freund's adjuvant, 10-fold concentrated, sterile toxoided Clostridium difficile antigen was prepared according to Example 6.

Two different pharmaceutical compositions using the toxoided antigen will be described. A first composition was prepared including a Clostridium difficile antigen without the Candida albicans antigen (termed CDT) by emulsifying a ten-fold concentrated, sterile toxoided Clostridium difficile antigen with an equal volume of incomplete Freund's adjuvant (Becton Dickinson and Company; Franklin Lakes, N.J.) to a final volume of 1.5 ml. A second composition was prepared including a Clostridium difficile antigen and a Candida albicans antigen (termed CAD) by emulsifying a ten-fold concentrated, sterile toxoided Clostridium difficile antigen with an equal volume of incomplete Freund's adjuvant (Becton Dickinson and Company; Franklin Lakes, N.J.) to a volume of 1.5 ml. Then, 1.5 ml of whole cell Candida albicans antigen emulsion were added to the Clostridium difficile preparation.

### Example 8

Bovine Immunization Studies

Gestating Holstein cows were maintained according to generally accepted dairy management practices at commercial Grade A dairy farms in Minnesota. Twenty-two cows were immunized subcutaneously with the CAD composition prepared as in Example 7. One-hundred seventeen cows were immunized subcutaneously with the CDT composition prepared as in Example 7. Each animal received 3 to 4 immunizations beginning approximately 60 days prepartum, and the immunizations were administered at approximately equal intervals. Colostrum samples were obtained from the third milking postpartum. The samples were frozen within one hour of collection for shipment to an analytical laboratory and remained frozen until analysis.

Specific antibody activity was monitored by enzyme immunoassay. Bovine IgG levels to Clostridium difficile were measured by enzyme-linked immuno-sorbent assay (ELISA) using a modification of the method described by Kelly et al., Gastroenterology, 102: 35-40 (1992). The coating antigen used to determine IgG titers was identical to the toxoided Clostridium difficile antigen preparation used to immunize the cows. Microtiter plates (Immunlon II; DYNEX Technologies, Inc.; Chantilly, Va.) were coated with 10 μg protein per ml in carbonate buffer at pH 9.6. 100 μL of the coating was applied to each well. The plates were incubated for 2 hours at 37° C. and then incubated overnight at 4° C. The plates were washed with phosphate buffered saline containing 0.05% Tween 20 (PBS-T) between each incubation step. To block the plates, 100 μL of 2% human serum albumin (ICN Biomedicals, Inc.; Costa Mesa, Calif.) in PBS was added to each well and the plates were incubated at room temperature for one hour.

Specific antibody activity of the individual colostrum samples was determined using doubling dilutions of colostrum for the initial binding activity. Horseradish peroxidase-labeled goat anti-bovine IgG (Kirkegaard & Perry Laboratories; Gaithersburg, Md.) was added as the secondary antibody (at 0.2 μg per ml in PBS with 2% human serum albumin) and incubated at 37° C. for one hour. TMB microwell peroxidase substrate (Kirkegaard & Perry Laboratories; Gaithersburg, Md.) was added as the substrate (100 μL per well) and stopped after 2 to 5 minutes with an equal volume of 1M phosphoric acid. The optical density was then read at 450 nm with 630 nm as a reference using an automated photometer (DYNEX Technologies, Inc.; Chantilly, Va.). The final titer determined for each sample was the last dilution point with an optical density greater than the standard non-immune colostrum control sample. Group immune responses were compared using a geometric mean titer (GMT) calculation according to Steel & Torrie (eds.), Principles and Procedures of Statistics (McGraw-Hill, NY, 1980).

Cows immunized with the CDT composition showed a GMT of 0.5596. Cows immunized with the CAD composition showed a GMT of 1.414. Thus, cows immunized with the pharmaceutical composition of the present invention showed a significant improvement in immune response.

### Example 9

Nutraceutical Food Preparation

A clinical nutritional beverage containing immunoglobulins collected from bovine milk or colostrum, alone or in combination with other biologically active components, is useful for the promotion of health.

One example of a nutritional beverage can be prepared by adding a composition of immunoglobulins, according to the invention, to a liquid, such as, for example, milk or fruit juice. A composition of immunoglobulins can be prepared by first preparing a CAD composition according to Example 7 and then administering the CAD composition to a gestating Holstein cow according to Example 8. The immunoglobulins that are reactive with a Clostridium difficile antigen are collected from colostrum according to Example 6 and U.S. Pat. No. 5,773,000 to Bostwick et al.

A clinical nutritional beverage can be prepared by adding 50-100 mg of a composition of immunoglobulins powder to 8 oz. of a liquid, such as, for example, milk or fruit juice.

From the foregoing detailed description and examples, it will be evident that modifications and variations can be made in the products and methods of the invention without departing from the spirit or scope of the invention. Therefore, it is intended that all modifications and verifications not departing from the spirit of the invention come within the scope of the claims and their equivalents.

What is claimed is:

1. A pharmaceutical composition comprising a whole cell Candida species antigen and a bacterial antigen.

2. The pharmaceutical composition of claim 1, wherein the bacterial antigen comprises a gram-positive bacterium.

3. The pharmaceutical composition of claim 1, wherein the bacterial antigen comprises a gram-negative bacterium.

4. The pharmaceutical composition of claim 1, further comprising at least one adjuvant.

5. The pharmaceutical composition of claim 4, wherein the adjuvant is incomplete Freund's adjuvant.

6. The pharmaceutical composition of claim 1, wherein the whole cell Candida species antigen is inactivated whole cell Candida albicans and the bacterial antigen is derived from Clostridium difficile.

7. The pharmaceutical composition of claim 6, wherein the Clostridium difficile antigen is toxoided.

8. The pharmaceutical composition of claim 6, wherein the Clostridium difficile antigen is derived from a Clostridium difficile organism deposited under ATCC accession no. 202193.

9. An immunoglobulin composition comprising immunoglobulins collected from an animal immunized with a pharmaceutical composition comprising a whole cell Candida species antigen and a bacterial antigen.

10. The immunoglobulin composition of claim 9, wherein the bacterial antigen is derived from Clostridium difficile.

11. The immunoglobulin composition of claim 10, wherein the Clostridium difficile antigen is toxoided.

12. The immunoglobulin composition of claim 10, wherein the Clostridium difficile antigen is derived from a Clostridium difficile organism deposited under ATCC accession no. 202193.

13. The immunoglobulin composition of claim 9, wherein the animal from which the immunoglobulins are collected is a cow.

14. The immunoglobulin composition of claim 13, wherein the immunoglobulins are collected from colostrum from the cow.

15. A nutraceutical comprising:
a nutrient component; and
an immunoglobulin composition, wherein the immunoglobulin composition is prepared according to a method of:
(i) administering to an animal a pharmaceutical composition comprising a whole cell Candida species antigen and a selected antigen;
(ii) collecting immunoglobulins from the animal; and
(iii) adding the collected immunoglobulins to the nutrient component to prepare a nutraceutical.

16. The nutraceutical of claim 15, wherein the Candida species is Candida albicans.

17. The nutraceutical of claim 15, wherein the selected antigen is derived from an organism selected from the group consisting of Cryptosporidium species and Clostridium species.

18. The nutraceutical of claim 17, wherein the selected antigen is derived from Clostridium difficile.

19. The nutraceutical of claim 18, wherein the Clostridium difficile antigen is toxoided.

20. The nutraceutical of claim 18, wherein the Clostridium difficile antigen is derived from a Clostridium difficile organism deposited under ATCC accession no. 202193.

21. The nutraceutical of claim 17, wherein the selected antigen is derived from Cryptosporidium parvum.

22. The nutraceutical of claim 15, wherein the animal is a cow.

23. The nutraceutical of claim 22, wherein the immunoglobulins are collected from colostrum from the cow.

24. A process for preparing a nutraceutical comprising:
(i) administering to an animal a pharmaceutical composition comprising a whole cell Candida species and a selected antigen;
(ii) collecting immunoglobulins from the animal; and
(iii) adding the collected immunoglobulins to a nutrient component to prepare a nutraceutical.

25. The process of claim 24, wherein the pharmaceutical composition comprises a whole cell Candida albicans.

26. The process of claim 24, wherein the selected antigen is derived from an organism selected from the group consisting of Cryptosporidium species and Clostridium species.

27. The process of claim 26, wherein the selected antigen is derived from Cryptosporidium parvum.

28. The process of claim 26, wherein the selected antigen is derived from Clostridium difficile.

29. The process of claim 28, wherein the Clostridium difficile antigen is toxoided.

30. The process of claim 28, wherein the Clostridium difficile antigen is derived from a Clostridium difficile organism deposited under ATCC accession no. 202193.

31. The process of claim 24, wherein the animal is a cow.

32. The process of claim 31, wherein the immunoglobulins are collected from colostrum from the cow.
